(19) United States
(12) Patent Application Publication
(10) Pub. No.: US 2023/0189841 A1
(43) Pub. Date: Jun. 22, 2023

(54) SYSTEMS OF ENZYMATIC TREATMENT FOR TENDERIZING MEAT ANALOG FOOD PRODUCTS

(71) Applicant: The Livekindly Company Switzerland GmbH, Pfaffikon (CH)

(72) Inventors: Julie Daoust, Los Angeles, CA (US); Shirin Madanshekaf, Dobbs Ferry, NY (US)

(21) Appl. No.: 17/556,290

(22) Filed: Dec. 20, 2021

(51) Int. Cl. A23J 3/26; C12N 9/28; A23J 3/14

(52) U.S. Cl. CPC A23J 3/26; C12N 9/2417; A23J 3/14; C12Y 302/01001

(57) ABSTRACT

Embodiments include systems and methods for texturizing and tenderizing plant protein meat analog food products with animal meat-like qualities using an enzymatic treatment of a High Moisture Extrusion (HME) extrudate. For example, automatically executing a recipe with a baseline plant protein meat analog food texture and a baseline plant protein meat analog food tenderness, using an automatic and dynamic enzymatic treatment for texturizing and tenderizing a plant protein meat analog food product to the baseline plant protein meat analog food texture and the baseline plant protein meat analog food tenderness thereby texturizing and tenderizing the plant protein meat analog food product with qualities to taste like animal meat by reducing a density of the plant protein meat analog food product by creating space between fibers of the plant protein meat analog food product resulting in an animal meat-like mouthfeel and taste for consumers.

### FIELD OF TECHNOLOGY

The present disclosure relates to methods for texturizing and tenderizing plant protein meat analog food products with animal meat-like qualities using an enzymatic treatment of a High Moisture Extrusion (HME) extrudate.

### SUMMARY

Embodiments of the present disclosure may include systems and methods for texturizing and tenderizing plant protein meat analog food products with animal meat-like qualities using an enzymatic treatment of a High Moisture Extrusion (HME) extrudate. In some aspects, the techniques described herein relate to a system for texturizing and tenderizing plant protein meat analog food products with animal meat-like qualities using an enzymatic treatment of a High Moisture Extrusion (HME) extrudate, the system including: a high moisture extrusion (HME) system including using an automatic and dynamic enzymatic treatment according to a recipe for texturizing and tenderizing a plant protein meat analog food product, the high moisture extrusion (HME) system including: a dry feed system, the dry feed system feeding plant protein into the high moisture extrusion (HME) system; a water feed system, the water feed system feeding water into the high moisture extrusion (HME) system to mix with the plant protein; a barrel system for mixing the plant protein and the water, the barrel system including: a shaft; at least one screw controlled by the shaft; and a heating system; a cooling die, the cooling die being maintained at a baseline cooling die temperature by the heating system and producing a High Moisture Extrusion (HME) extrudate; a vacuum tumbler for cooking the High Moisture Extrusion (HME) extrudate to the plant protein meat analog food product; an enzyme pump, the enzyme pump automatically and dynamically adding the enzymatic treatment according to the recipe to the vacuum tumbler with the High Moisture Extrusion (HME) extrudate; an electronic sensor system, the electronic sensor system electronically connected to the dry feed system, the water feed system, the barrel system, the cooling die, the vacuum tumbler, the enzyme pump; and a main operation panel, the main operation panel being electronically connected to the electronic sensor system, the main operation panel including: at least one processor; and a memory storing processor-executable instructions, wherein the at least one processor is configured to implement the following operations for the automatic and dynamic enzymatic treatment for texturizing and tenderizing the plant protein meat analog food product upon executing the processor-executable instructions: automatically executing, using the electronic sensor system, the recipe with a baseline plant protein meat analog food texture and a baseline plant protein meat analog food tenderness, using the automatic and dynamic enzymatic treatment for texturizing and tenderizing the plant protein meat analog food product to the baseline plant protein meat analog food texture and the baseline plant protein meat analog food tenderness thereby texturizing and tenderizing the plant protein meat analog food product with qualities to taste like animal meat by reducing a density of the plant protein meat analog food product by creating space between fibers of the plant protein meat analog food product resulting in an animal meat-like mouthfeel and taste for consumers of the plant protein meat analog food product.

In some embodiments, the techniques described herein relate to a system, wherein the enzymatic treatment includes a neutral protease enzyme.

In some embodiments, the techniques described herein relate to a system, wherein the neutral protease enzyme includes 0.25 percent to 1 percent (w/w) of the neutral protease enzyme to a weight of the High Moisture Extrusion (HME) extrudate.

In some embodiments, the techniques described herein relate to a system, wherein the enzymatic treatment includes an alpha-amylase enzyme.

In some embodiments, the techniques described herein relate to a system, wherein the alpha-amylase enzyme includes 0.1 percent to 1 percent (w/w) of the alpha-amylase enzyme to a weight of the High Moisture Extrusion (HME) extrudate.

In some embodiments, the techniques described herein relate to a system, wherein the enzymatic treatment includes a neutral protease enzyme and an alpha-amylase enzyme.

In some embodiments, the techniques described herein relate to a system, wherein a ratio of the neutral protease enzyme to the alpha-amylase enzyme is from 1 to 0.25 percent and 1 to 1.

In some embodiments, the techniques described herein relate to a system, wherein the automatic and dynamic enzymatic treatment for texturizing and tenderizing the plant protein meat analog food product further includes increasing the baseline plant protein meat analog food texture to an optimized plant protein meat analog food texture, the optimized plant protein meat analog food texture resulting optimized animal meat-like mouthfeel and taste for the consumers of the plant protein meat analog food product.

In some embodiments, the techniques described herein relate to a system, wherein the automatic and dynamic enzymatic treatment for texturizing and tenderizing the plant protein meat analog food product further includes decreasing the baseline plant protein meat analog food texture to an optimized plant protein meat analog food texture, the optimized plant protein meat analog food texture resulting optimized animal meat-like mouthfeel and taste for the consumers of the plant protein meat analog food product.

In some embodiments, the techniques described herein relate to a system, wherein the automatic and dynamic enzymatic treatment for texturizing and tenderizing the plant protein meat analog food product further includes increasing the baseline plant protein meat analog food tenderness to an optimized plant protein meat analog food tenderness, the optimized plant protein meat analog food tenderness resulting in optimized animal meat-like mouthfeel and taste for the consumers of the plant protein meat analog food product.

In some embodiments, the techniques described herein relate to a system, wherein the automatic and dynamic enzymatic treatment for texturizing and tenderizing the plant protein meat analog food product further includes decreasing the baseline plant protein meat analog food tenderness to an optimized plant protein meat analog food tenderness, the optimized plant protein meat analog food tenderness resulting in optimized animal meat-like mouthfeel and taste for the consumers of the plant protein meat analog food product.

In some embodiments, the techniques described herein relate to a method for texturizing and tenderizing plant protein meat analog food products with animal meat-like qualities using an enzymatic treatment of a High Moisture Extrusion (HME) extrudate, the method including: automatically executing, using an electronic sensor system, a recipe with a baseline plant protein meat analog food texture and a baseline plant protein meat analog food tenderness, using an automatic and dynamic enzymatic treatment for texturizing and tenderizing a plant protein meat analog food product to the baseline plant protein meat analog food texture and the baseline plant protein meat analog food tenderness thereby texturizing and tenderizing the plant protein meat analog food product with qualities to taste like animal meat by reducing a density of the plant protein meat analog food product by creating space between fibers of the plant protein meat analog food product resulting in an animal meat-like mouthfeel and taste for consumers of the plant protein meat analog food product.

In some embodiments, the techniques described herein relate to a method, wherein the enzymatic treatment includes a neutral protease enzyme.

In some embodiments, the techniques described herein relate to a method, wherein the neutral protease enzyme includes 0.25 percent to 1 percent (w/w) of the neutral protease enzyme to a weight of the High Moisture Extrusion (HME) extrudate.

In some embodiments, the techniques described herein relate to a method, wherein the enzymatic treatment includes an alpha-amylase enzyme.

In some embodiments, the techniques described herein relate to a method, wherein the alpha-amylase enzyme includes 0.1 percent to 1 percent (w/w) of the alpha-amylase enzyme to a weight of the High Moisture Extrusion (HME) extrudate.

In some embodiments, the techniques described herein relate to a method, wherein the enzymatic treatment includes a neutral protease enzyme and an alpha-amylase enzyme.

In some embodiments, the techniques described herein relate to a method, wherein a ratio of the neutral protease enzyme to the alpha-amylase enzyme is from 1 to 0.25 percent and 1 to 1.

In some embodiments, the techniques described herein relate to a non-transient computer-readable storage medium including instructions being executable by one or more processors to perform operations for texturizing and tenderizing plant protein meat analog food products with animal meat-like qualities using an enzymatic treatment of a High Moisture Extrusion (HME) extrudate, the operations including: automatically executing, using an electronic sensor system, a recipe with a baseline plant protein meat analog food texture and a baseline plant protein meat analog food tenderness, using an automatic and dynamic enzymatic treatment for texturizing and tenderizing a plant protein meat analog food product to the baseline plant protein meat analog food texture and the baseline plant protein meat analog food tenderness thereby texturizing and tenderizing the plant protein meat analog food product with qualities to taste like animal meat by reducing a density of the plant protein meat analog food product by creating space between fibers of the plant protein meat analog food product resulting in an animal meat-like mouthfeel and taste for consumers of the plant protein meat analog food product.

In some embodiments, the techniques described herein relate to a non-transient computer-readable storage medium, wherein the enzymatic treatment includes a neutral protease enzyme and an alpha-amylase enzyme.

### BRIEF DESCRIPTION OF THE DRAWINGS

The accompanying drawings, where like reference numerals refer to identical or functionally similar elements throughout the separate views, together with the detailed description below, are incorporated in and form part of the specification, and serve to further illustrate embodiments of concepts that include the claimed disclosure, and explain various principles and advantages of those embodiments.

The methods and systems disclosed herein have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

FIG. 1A shows a system for texturizing and tenderizing plant protein meat analog food products with animal meat-like qualities using an enzymatic treatment of a High Moisture Extrusion (HME) extrudate, according to various embodiments of the present technology.

FIG. 1B illustrates a top view of a system for vacuum cooking the High Moisture Extrusion (HME) extrudate with the enzymatic treatment, according to various embodiments of the present technology.

FIG. 2 illustrates a system configured for texturizing and tenderizing plant protein meat analog food products with animal meat-like qualities using an enzymatic treatment of a High Moisture Extrusion (HME) extrudate, according to various embodiments of the present technology.

FIG. 3 illustrates a method for texturizing and tenderizing plant protein meat analog food products with animal meat-like qualities using an enzymatic treatment of a High Moisture Extrusion (HME) extrudate, according to various embodiments of the present technology.

### DESCRIPTION OF EXEMPLARY EMBODIMENTS

A problem in the food processing industry is making plant-based food products taste like animal meat. For example, depending on the specific plant protein, some plant-based products may have a gritty texture or a flavor with bitter notes. In other words, a problem in the food processing industry is that plant-based food products are often made of soy, pea, or almond protein, and the like, all of which have different cell structure than traditional animal meat or dairy that may lead to food products made from plant proteins that have a different texture, which results in a differ mouthfeel and taste for consumers. The process of making plant-based food products taste like animal meat is texturing and tenderizing. Texturizing and tenderizing, in other words, is providing plant-based food products with qualities to taste like animal meat for consumers.

The present technology solves this problem of texturizing and tenderizing plant-based food products with animal meat-like qualities by using an enzymatic treatment during food processing resulting in plant-based food products with animal meat mouthfeel and taste for consumers. For example, during High Moisture Extrusion (HME) food processing, a solution of enzymatic treatment is used for texturizing and tenderizing the plant-based food products. For example, plant-based food products produced using High Moisture Extrusion (HME) have a cell structure of with fibers that are more densely packed than the fibers of animal meat. Thus, the fibers of plant-based food products need to be separated (i.e., “opened-up”) to taste like animal meat for consumers. The enzymatic treatment solution of the present technology texturizes and tenderizes, in other words, provides plant-based food products with qualities to taste like animal meat by reducing a density of plant-based food products and creating space between fibers of the of plant-based food products resulting in a taste that is like animal meat.

Various approaches use unique methods of meat analog processing including 3D printing, electrostatic spinning, thermosonic technology and ultrasound may be used to solve the problem of texturizing and tenderizing plant-based food products with animal meat-like qualities. For example, the following patent applications are incorporated by reference in their entireties including all references cited therein.

Use of Extrusion in the Meat Analog Manufacturing Process.

Various approaches use an extrusion process to solve the problem of texturizing and tenderizing plant-based food products with animal meat-like qualities. For example, the following patent applications are incorporated by reference in their entireties including all references cited therein: WO Patent Application Publication No. WO2020180651A1 (KELLOGG) titled “Vegetable based snack stick” uses extrusion for making encased vegetable based jerky (e.g., hydrating texturized vegetable protein mixture to fibrous mass, hydrating gel forming protein isolate to protein gel, mixing gel with the mass to mixed mass and extruding mass to casing. US Patent Application Publication No. US20170303558A1 (FRAUNHOFER ([DE])), titled “Extruded plant protein product with coloring plant ingredients and production method” uses extrusion for the extruded plant protein product providing specific texture and excellent mouthfeel to the food products. US Patent Application Publication No. US20190045809A1 (SUNFED ([NZ]) titled “Meat substitute” uses extrusion for the extruded meat substitute composition producing an extruded meat substitute with improved characteristics of texture, resilience, moisture and mouthfeel over existing products, and the product is a low allergen product, preferably formed without soy or without soy and gluten, and suitable for both vegetarian and nonvegetarian consumers, and has fibrous quality of meat, while retaining moisture and flavor during cooking. WO Patent Application Publication No. WO2019143859A1 (HERSHEY ([US]) titled “Formulations and methods of preparing products with meat-like texture with plant-based protein sources” uses Extrusion: by preparing products with meat-like texture with plant-based protein sources (e.g., gluten-free) by combining plant-based protein sources, heating and extruding composition, cooling composition under laminar flow, and drying extrudate. The plant-based products have anisotropically fibrous meat-like texture, shelf stability, tender meat-like texture, flavor similar to meat, and texture similar to meat; and are free of soy proteins, gluten-free, and free of the beany off-note flavor commonly found in plant protein-based formulations. CN Patent Application Publication No. CN112006152A (NORTHEAST AGRICULTURAL UNIVERSITY ([CN])) titled “Method for preparing vegetarian meat by using soybean protein isolate” uses extrusion, but also shearing, and aims to produce thicker (muscle-like) meat analog. WO Patent Application Publication No. WO2020208104A1 (NESTLE ([CH]) INSTITUT DE RECHERCHE POUR LAGRICULTURE LALIMENTATION & LENVIRONNEMENT INRAE ([FR]) UNIVERSITE DE MONTPELLIER 2 ([FR]) titled “Meat analogues and meat analogue extrusion devices and methods” uses Extrusion and shearing for providing Meat analogue comprising a macrostructure of connected sheared fibers oriented substantially parallel to one another; and gaps positioned between the sheared fibers, where the macrostructure does not comprise meat, and the macrostructure comprises a vegetable protein. The meat analogues exhibits fibrous macrostructure and voids in the macrostructure where fat is injected into the voids. WO Patent Application Publication No. WO2020037368A1 (V2 FOOD ([AU]) titled “Food ingredients” uses Low Moisture extrusion: for Preparing protein-carbohydrate complex food ingredient used in meat mimetic food product (e.g., meatballs or nuggets), by mixing protein source and carbohydrate source in extruder, and subjecting mixture to low moisture extrusion.

Various approaches use unique methods of meat analog processing including 3D printing, electrostatic spinning, thermosonic technology and ultrasound may be used to solve the problem of texturizing and tenderizing plant-based food products with animal meat-like qualities. For example, the following patent applications are incorporated by reference in their entireties including all references cited therein: WO Patent Application Publication No. WO2020152689A1 (REDEFINE MEAT LTD) titled MEAT ANALOGUES AND METHODS OF PRODUCING THE SAME uses 3D printed meat slab analogues (protein-based and fat-based component). WO Patent Application Publication No. WO2020030628A1 (NOVAMEAT TECHNOLOGY ([ES])) titled Process of manufacturing edible microextruded product comprising protein, composition thereby obtained and the use thereof 3D printing: Abstract: The invention relates to edible microextruded products with compressive and tensile Young's moduli resembling the mechanical properties of meat, said edible products comprising several layers of microextruded elements made of a viscoelastic composition, said viscoelastic composition comprising in an appropriate edible solvent, high amounts of protein and an edible pseudoplastic polymer. The invention discloses also particular processes for obtaining such edible micro extruded products, in particular a 3D printing method. Particular uses of edible products are also listed, including the use as meat-replacers. The invention discloses also new edible viscoelastic compositions comprising proteins and pseudoplastic polymers. CN Patent Application Publication No. CN111869787A (NORTHEAST AGRICULTURAL UNIVERSITY ([CN])) titled Preparation method of high-fibrosis soybean meat-like product uses both Ultrasonic modification and electrostatic spinning on the soybean silk protein for preparing high-fiber soybean meat-like products, by placing intact soybean silk protein in dehydrator, carrying out ultrasonic modification treatment on the dehydrated soybean silk protein, mixing, performing electrospinning treatment and sterilizing product. The power of the ultrasonic crusher is 300-400 w, and the ultrasonic treatment time is 15-20 minutes. The conditions of electrostatic spinning: temperature is 35-40° C. and relative humidity is 48-50%. The parameters of the electrostatic spinning device: the voltage is 15-20 kV, and the solution flow rate is 5-6 ml/minute. The pulse energy of the pulsed strong light sterilization is 400-500 J, the pulse distance is 10-13 cm, and the pulse time is 50-80 seconds. CN Patent Application Publication No. CN111903833A (JIANGNAN UNIVERSITY ([CN])) titled Preparation method of plant protein powder based on thermosonic technology, product and application thereof provides methods of plant protein powder based on a thermosonic technology. For example, the invention discloses a preparation method of plant protein powder based on a thermosonic technology, a product and application thereof, comprising the steps of dispersing plant protein in purified water to prepare a plant protein aqueous solution, and controlling the concentration of the plant protein in the plant protein aqueous solution to be less than or equal to 20 percent; carrying out thermal ultrasonic treatment on the plant protein aqueous solution to obtain a protein solution containing aggregates, wherein the ultrasonic power is 10-800 W, the frequency is 10-30 kHz, the amplitude is 10%-40%, the temperature is 30-90° C., and the time is 5-120 min; the protein solution containing the aggregate is subjected to spray drying treatment to prepare soluble plant protein isolate powder, the content of the obtained protein powder aggregate is 5-30%, and the gel temperature is 50-100° C. The content of the vegetable protein aggregate is 5-30%, the protein powder can form a heat irreversible gel after being heated and induced, the gel has high elasticity and good water and oil holding capacity, the texture and the taste of the finally prepared vegetable protein meat are close to real meat

Methods for Meat Analog using Hydrocolloids or Emulsions.

Various approaches use a hydrocolloids or emulsions process to solve the problem of texturizing and tenderizing plant-based food products with animal meat-like qualities. For example, the following patent applications are incorporated by reference in their entireties including all references cited therein: US Patent Application Publication No. US20150351427A1 titled Pulse Tex, Rovita Method for producing meat substitute products provides methods for producing meat substitute products in which a mixture of water and a vegetable fat or oil with a protein, sodium alginate and methylcellulose is processed by shearing to form a stable emulsion and a colloidal solution of divalent metal cations and micellar casein is added to the emulsion to initiate fibre formation, wherein in order to monitor the reaction rate, the quantity of added divalent metal cations is not sufficient to initiate fibre formation but is selected so that it only leads to the initiation of fibre formation in combination with the calcium fraction of the micellar casein. The method achieves controlled and uniform fiber formation. WO Patent Application Publication No. WO2018177717A1 (Unilever) titled Meat alternative comprising aqueous gelling composition uses an aqueous gelling composition to enhance the juiciness of meat analogues. (“The inventors have found that such a meat analogue can be prepared by combining proteinaceous fibers of non-meat origin with an aqueous gelling composition that contains xanthan gum and galactomannan”.) U.S. Pat. No. 6,379,738B1 (Nestle) titled Meat emulsion product provides methods for producing meat emulsion products (e.g., sausage and Bologna). Meat emulsion products are produced that have realistic fiber definition. The meat emulsion product comprises a body member including protein and fat and having a plurality of linear strands of fiber-like material affording the meat emulsion product a realistic meat-like appearance. The meat emulsion product is not pasty, mushy or brittle and has realistic meat-like appearance. The integrity and shape of the product are retained when subjected to commercial canning and sterilization processes.

Mycoprotein or Fungal Mycelium for Meat Analogs.

Various approaches use mycoprotein to solve the problem of texturizing and tenderizing plant-based food products with animal meat-like qualities. For example, the following patent applications are incorporated by reference in their entireties including all references cited therein: US Patent Application Publication No. US20200157506A1 (ECOVATIVE DESIGN ([US])) titled Methods of Generating Mycelial Scaffolds and Applications Thereof provides a very broad disclosure on mycelium scaffolds (e.g., mycological biopolymer, including for biomedical applications): non-animal bacon-like product and meat alternative. CN Patent Application Publication No. CN110623081A (GUIZHOU BEZON FOOD INDUSTRY ([CN])) titled Dry-mushroom vegetarian diced meat and processing method thereof provides dried mycelium diced meat comprises shiitake mushrooms, coprinus, oyster mushrooms, agrocybe, straw mushrooms, real shiitake mushrooms, laver, spices, glutinous rice flour, soy protein. CN Patent Application Publication No. CN104171016A (CHEN RONG) titled Chicken-leg-shaped vegetable dish and preparation method thereof used Veggie chicken leg comprising filling of 70-80 pts. wt. Hericium erinaceus, 5-10 pts. wt. mushroom, 5-10 pts. wt. soybean protein and 15-35 mass pts. wt. seasoning material. CN Patent Application Publication No. CN105495561A (JIANGSU FOOD & PHARMACEUTICAL SCIENCE COLLEGE) titled Vegetarian drumstick and preparation method thereof provides a vegetarian chicken drumstick including composition from mushroom Coprinus comatus. CA Patent Application Publication No. CA3100909A1 (EMERGY ([US])) titled methods for growing fungal mycelium and forming edible products therefrom provides methods of growing fungal mycelium and forming edible food products. The mycelium is concentrated to obtain a fibrous mycelium mass having a protein content of greater than 40 wt % of a dry mass of the mycelium. Food additives can be added to the fibrous mycelium mass to produce a biomass. The biomass can be formed into an edible food product. The biomass can be formed into an edible meat substitute product. WO Patent Application Publication No. WO2016120594A1 (Marlow Foods (Quorn, now Monde Nissin)) titled Edible Fungi provides a meat analog brand that uses mycoprotein derived from Fusarium venenatum (grown by fermentation) as the main ingredient for their product. Incorporated by reference is U.S. Pat. No. 4,555,485A that describes the use of mycoprotein as an ingredient for meat analogs by Marlow Foods (Robert A. Marsh). WO Patent Application Publication No. WO2016120594A1 provides an edible fungi formulation that is vegan. Usually the mycoprotein-based product is dried and mixed with egg white, which acts as a binder. To make this edible fungi formulation vegan, calcium ions are used instead. The patent describes that “the addition of calcium cations (via calcium chloride and calcium acetate) to the ingredients described in the examples produce a rise in firmness and produces acceptable quality of the product produced. It is believed the calcium cations interact with the mycoprotein paste to increase its firmness and strength.” Furthermore, if calcium acetate is used, there is no off taste generated.

MycoTechnology.

Various approaches use a mycotechnology process (i.e., harnessing the metabolic engine of mushrooms, known as mycelium, to discover naturally transformative ingredients that solve the food industry's biggest challenges. Mycelia provide the mushroom with the food it needs to survive and flourish. Unlike humans, or even other fungi, mushrooms digest their food externally by secreting enzymes into their external environment; enzymes that break down complex organic matter into simpler nutrients—like sugars, nitrates and phosphates) to solve the problem of texturizing and tenderizing plant-based food products with animal meat-like qualities. For example, the following patent applications are incorporated by reference in their entireties including all references cited therein: WO Patent Application Publication No. WO2020232347A1 (MYCOTECHNOLOGY ([US])) titled Myceliated protein compositions having improved texture and methods for making provides methods for preparing a protein food product (i.e., meat analog) used as protein food for human or animal consumption. The substrate includes at least 50 wt. % protein isolate or concentrate by dry weight. The sterilized substrate is inoculated with a filamentous fungal culture in solid state fermentation conditions. The filamentous fungal culture and the sterilized substrate is cultured, where the filamentous fungal culture grows hyphae and forms a mycelial network to form the protein food product. The protein food product is more cohesive than a non-myceliated control substrate after cooking, and/or has more spring than a non-myceliated control substrate after cooking, and/or has more juiciness than a non-myceliated control substrate after cooking. The protein food product has increased desirable flavors and/or reduced undesirable aromas compared to a non-myceliated control substrate. WO Patent Application Publication No. WO2020092306A1 (MYCOTECHNOLOGY ([US])) titled Myceliated vegetable protein and food compositions comprising same provides methods for a food composition (including meat analog as one of the examples) with: reduced undesirable flavors and reduced undesirable aromas due to use of myceliated high-protein food products as compared to use of similar high-protein material that is not myceliated; and low-cost vegetarian protein sources. WO Patent Application Publication No. WO2020154634A1 (MYCOTECHNOLOGY) ([US]) titled Methods for the production and use of myceliated amino acid-supplemented food compositions provides the myceliated amino acid-supplemented high-protein food product has reduced bitterness and/or reduced volatile amino-acid-derived aroma compared to the high-protein amino acid-supplemented material that is not myceliated.

Freeze Structuring.

Various approaches use a freeze structuring process to solve the problem of texturizing and tenderizing plant-based food products with animal meat-like qualities. For example, the following non-patent literature document is incorporated by reference in its entirety including all references cited therein: Yuliarti et al., Structuring the meat analogue by using plant-based derived composites, Journal of Food Engineering, Volume 288, January 2021, 110138. A freeze structuring process is disclosed to form wheat/pea nuggets that were analyzed and compared to real chicken. 13% wheat/4% pea best emulated chicken. Achieves fibrous and layered structure by freezing of a protein emulsion. Moreover, the subsequent removal of ice crystals generates a porous and fibrous microstructure similar to that of animal meat muscles which comprised numerous parallel and highly connected sheet-like proteins. Moreover, Dekkers et al., Structuring processes for meat analogues, Trends in Food Science & Technology, Volume 81, November 2018, pp. 25-36, is incorporated by reference herein for a detailed discussion of the freeze structuring process.

Mechanical Elongation and Antisolvent Precipitation.

Various approaches use a Mechanical Elongation and Antisolvent Precipitation to solve the problem of texturizing and tenderizing plant-based food products with animal meat-like qualities. For example, the following non-patent literature document is incorporated by reference in its entirety including all references cited therein: Mattice et al., Comparing methods to produce fibrous material from zein, Food Research International, Volume 128, February 2020, 208804, and Mattice, Characterizations of Zein: Evaluating Functionality and Potential uses in Food Structuring, Ph.D. thesis at University of Guelph, Guelph, Ontario, Canada, disclose three methods to produce fibrous material from zein, either as fibrous networks or individual protein fibers, for the purpose of providing a fibrous structure to whole-tissue meat analogues. These methods are electrospinning; antisolvent precipitation of zein from ethanol using water; and mechanical elongation of self-assembled zein networks. When incorporated into model meat analogue soy protein isolate gels, similar textural properties to whole meat chicken were achieved by mechanical elongation and increases in ductility were achieved through antisolvent precipitation from ethanol.

Intermediate Moisture Extrusion.

Various approaches use Intermediate Moisture Extrusion to solve the problem of texturizing and tenderizing plant-based food products with animal meat-like qualities. For example, the following non-patent literature document is incorporated by reference in its entirety including all references cited therein: a Korean research center at Konju National University conducted a 2018 study to determine if texturized vegetable protein transformed from plant protein-based ingredient can impart fibrous structure like muscle meat. The study demonstrated that some textural and chemical characteristics (e.g., nitrogen solubility and integrity index; chewiness; cutting strength; non-uniform fiber structure) of texturized vegetable protein under intermediate moisture extrusion exhibited the most similarity to chicken meat. (Samard et al., A comparison of physicochemical characteristics, texture, and structure of meat analogue and meats, Journal of the Science of Food and Agriculture, Volume 99, Issue 6, April 2019, pp. 2708-2715).

Citrus Fiber Additives to Improve Meat Alternative Properties.

Various approaches use a Citrus Fiber Additives to Improve Meat Alternative Properties process to solve the problem of texturizing and tenderizing plant-based food products with animal meat-like qualities. For example, the following non-patent literature document is incorporated by reference in its entirety including all references cited therein: Fiberstar offers a Citri-Fi citrus fiber one-hundred series product line that performs several functions in meat alternatives, including adding juiciness, sizzling during cooking, thickening oil and binding fat. These attributes are a result of Citri-Fi's abilities to hold large quantities of both water and oil. The amounts or ratios of water and oil can be adjusted so that the amount bound and/or released during cooking targets the desired effect Fiberstar also offers the new Citri-Fi TX texturizing citrus fiber for binding, strengthening and adding texture Citri-Fi TX can be used to replace gluten and bind proteins together both during cold forming and cooking.

High Shear Spinning.

Various approaches use a High Shear Spinning process to solve the problem of texturizing and tenderizing plant-based food products with animal meat-like qualities. For example, the following non-patent literature document is incorporated by reference in its entirety including all references cited therein: Cybercolloids has developed a high shear spinning/mixing system to texturize plant-based proteins into meat-like fibers. The process is reportedly less expensive than extrusion and could be more efficient at producing large quantities of plant-based meat. The process is based on a precipitation mechanism using equipment from another manufacturing industry, combining plant-based protein powder with water and an unspecified binder. For example, U.S. Pat. No. 4,226,576A is incorporated by reference and covers high speed spinning techniques for forming texturized monofilament protein product having a texture and mouth feel simulating animal meat.

Electrospinning of Fibrous Proteins.

Various approaches use Electrospinning of Fibrous Proteins process to solve the problem of texturizing and tenderizing plant-based food products with animal meat-like qualities. For example, the following non-patent literature document is incorporated by reference in its entirety including all references cited therein: Nieuwland et al., Food-grade electrospinning of proteins, Innovative Food Science & Emerging Technologies, Volume 20, October 2013, pp. 269-275. A processes for food-grade electrospinning of proteins using gelatin as a carrier to produce thin fibrils as building blocks for texturally interesting meat analogs is disclosed. The spinning of proteins is very difficult, and most proteins cannot be spun under food-grade conditions. Only two proteins are known to spin under food-grade conditions—zein spun from ethanol and gelatin from warm water. The study looked at using one of these proteins as a carrier for other proteins. With gelatin, the researchers determined that it was possible to electrospin a range of globular proteins, showing for the first time that electrospinning of food-grade globular proteins was possible.

Shear Cell Technology.

Various approaches use Shear Cell Technology to solve the problem of texturizing and tenderizing plant-based food products with animal meat-like qualities. For example, the following non-patent literature document is incorporated by reference in its entirety including all references cited therein: Schreuders et al., Comparing structuring potential of pea and soy protein with gluten for meat analogue preparation, Journal of Food Engineering, Volume 261, November 2019, Pages 32-39. The references discloses shear cell formation of chicken fiber fillet analog meat from pea protein blends. Their research found that blends of pea protein isolate and wheat gluten can create fibrous meat analogs with a matrix strength that is similar to that of cooked chicken meat.

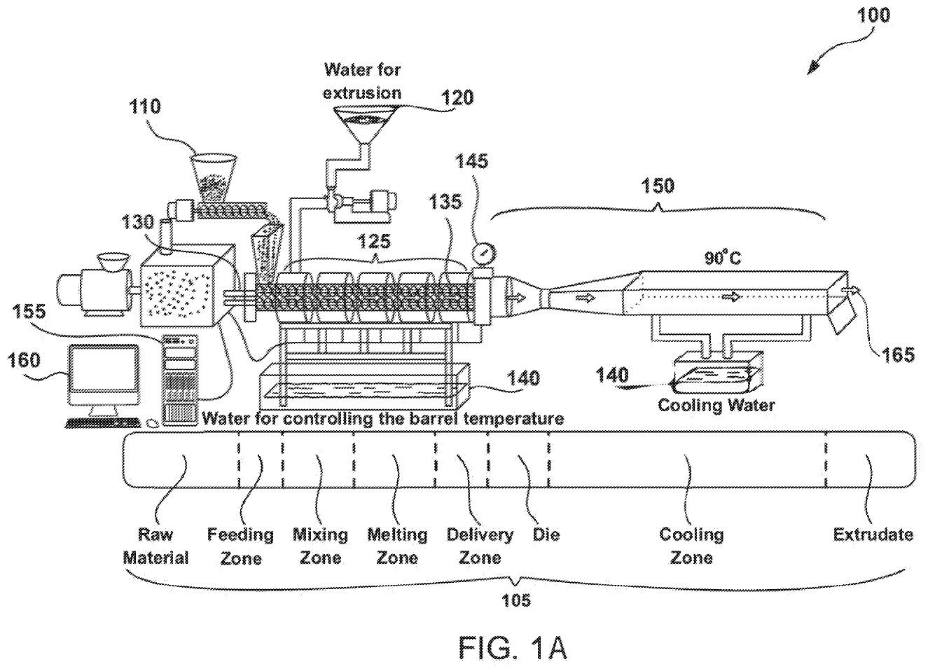

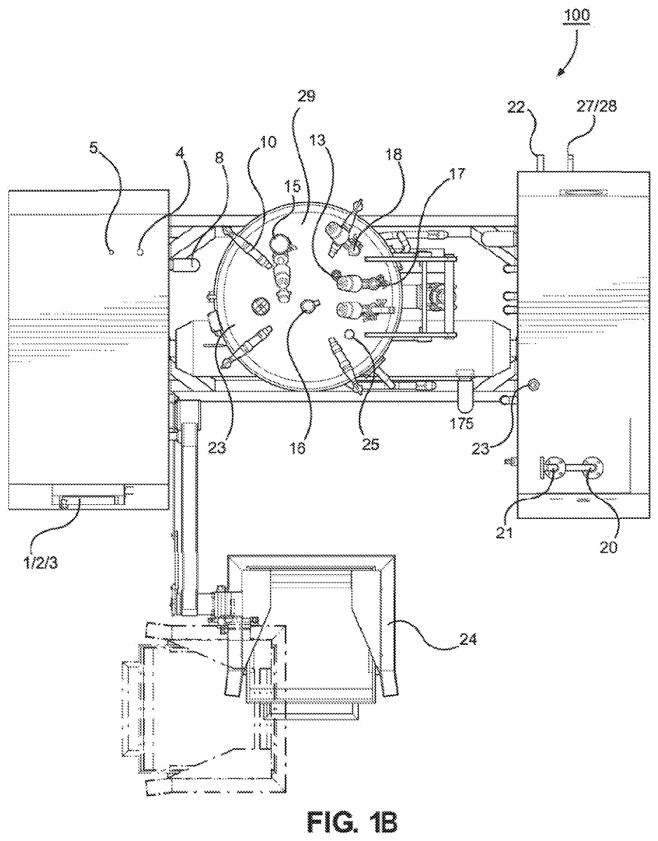

FIG. 1A for texturizing and tenderizing plant protein meat analog food products with animal meat-like qualities using an enzymatic treatment of a High Moisture Extrusion (HME) extrudate, according to various embodiments of the present technology.

A system for texturizing and tenderizing plant protein meat analog food products with animal meat-like qualities using an enzymatic treatment of a High Moisture Extrusion (HME) extrudate, the system comprising: a high moisture extrusion (HME) system 100 including using an automatic and dynamic enzymatic treatment according to a recipe for texturizing and tenderizing a plant protein meat analog food product, the high moisture extrusion (HME) system 100 comprising: a dry feed system 110, the dry feed system 110 feeding plant protein into the high moisture extrusion (HME) system; a water feed system 120, the water feed system 120 feeding water into the high moisture extrusion (HME) system 100 to mix with the plant protein; a barrel system 125 for mixing the plant protein and the water, the barrel system 125 comprising: a shaft 130; at least one screw 135 controlled by the shaft 130; and a heating system 140 (e.g., heating water and cooling water); a cooling die 150, the cooling die 150 being maintained at a baseline cooling die temperature by the heating system 140 and producing a High Moisture Extrusion (HME) extrudate 165; a vacuum tumbler 29 (e.g., vacuum tumbler 29 shown in FIG. 1B) for cooking the High Moisture Extrusion (HME) extrudate 165 to the plant protein meat analog food product; an enzyme pump 175 (shown in FIG. 1B), the enzyme pump 175 automatically and dynamically adding the enzymatic treatment according to the recipe to the vacuum tumbler 29 with the High Moisture Extrusion (HME) extrudate 165; an electronic sensor system 145, the electronic sensor 145 system electronically connected to the dry feed system 110, the water feed system 120, the barrel system 125, the cooling die 150, the vacuum tumbler 29, the enzyme pump 175; and a main operation panel 155, the main operation panel 155 being electronically connected to the electronic sensor system 145, the main operation panel 155 comprising: at least one processor; and a memory storing processor-executable instructions, wherein the at least one processor is configured to implement the following operations for the automatic and dynamic enzymatic treatment for texturizing and tenderizing the plant protein meat analog food product upon executing the processor-executable instructions: executing automatically, using the electronic sensor system 145, the recipe with a baseline plant protein meat analog food texture and a baseline plant protein meat analog food tenderness, using the automatic and dynamic enzymatic treatment for texturizing and tenderizing the plant protein meat analog food product to the baseline plant protein meat analog food texture and the baseline plant protein meat analog food tenderness thereby texturizing and tenderizing the plant protein meat analog food product with qualities to taste like animal meat by reducing a density of the plant protein meat analog food product by creating space between fibers of the plant protein meat analog food product resulting in an animal meat-like mouthfeel and taste for consumers of the plant protein meat analog food product.

FIG. 1B illustrates a top view of a system for vacuum cooking the High Moisture Extrusion (HME) extrudate 165 with the enzymatic treatment, according to various embodiments of the present technology. FIG. 1B shows the following elements: main operation panel 1 (e.g., may include a touch screen user interface), main switch 2, emergency stop 3, a power inlet 4, an air inlet 5, mixing motor cable 8, pneumatic cylinder 10, a steam injector 13 (e.g., a steam lance), a first safety valve 14, a second safety valve 15, a vacuum sensor 16, vacuum valve 17 (e.g., a pneumatic vacuum valve), pneumatic release valve 18, direct steam 20 (e.g., flange), steam double jacket 21, condensate return 22 (e.g., thread may be 1 inch in diameter), supply water 23 (e.g., hose nipple may be 0.5 inches in diameter), foldable lift 24, dosing steam nozzle 25 (e.g., dosing steam nozzle may be 0.5 inches in diameter), water ring pump inflow 27 (e.g., thread may be 0.5 inches in diameter), water ring pump return 28 (e.g., thread may be ¾ inches), and vacuum tumbler 29. In some instances, the steam double jacket 21 is heated by steam, and the steam double jacket 21 is used to heat the vacuum tumbler 29 during vacuum cooking. U.S. patent application Ser. No. 17/366,720, filed on Jul. 2, 2021, titled “Systems and Methods for Vacuum Cooking” shows additional details regarding systems and methods of vacuum cooking and is incorporated by reference herein for all purposes including all references cited therein.

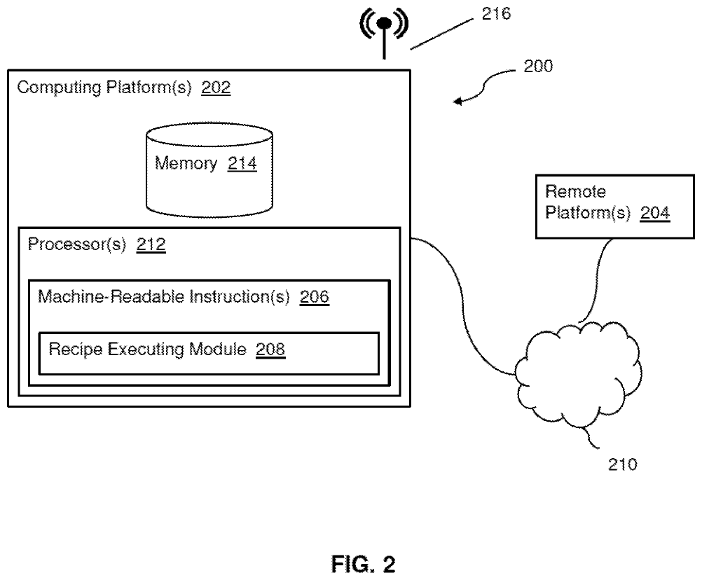

FIG. 2 illustrates a system configured for texturizing and tenderizing plant protein meat analog food products with animal meat-like qualities using an enzymatic treatment of a High Moisture Extrusion (HME) extrudate, in accordance with one or more embodiments. In some cases, system 200 may include one or more computing platforms 202. The one or more computing platforms 202 may be communicably coupled with one or more remote platforms 204. In some cases, users may access the system 200 via remote platform(s) 204.

The one or more computing platforms 202 may be configured by machine-readable instructions 206. Machine-readable instructions 206 may include modules. The modules may be implemented as one or more of functional logic, hardware logic, electronic circuitry, software modules, and the like. The modules may include one or more of recipe executing module 208, and/or other modules.

Recipe executing module 208 may be configured to execute automatically, using an electronic sensor system, a recipe with a baseline plant protein meat analog food texture and a baseline plant protein meat analog food tenderness, using an automatic and dynamic enzymatic treatment for texturizing and tenderizing a plant protein meat analog food product to the baseline plant protein meat analog food texture and the baseline plant protein meat analog food tenderness thereby texturizing and tenderizing the plant protein meat analog food product with qualities to taste like animal meat by reducing a density of the plant protein meat analog food product by creating space between fibers of the plant protein meat analog food product resulting in an animal meat-like mouthfeel and taste for consumers of the plant protein meat analog food product.

In some cases, the enzymatic treatment comprises a neutral protease enzyme, the neutral protease enzyme comprises 0.25 percent to 1 percent of the neutral protease enzyme to a weight of the High Moisture Extrusion extrudate and the enzymatic treatment comprises an alpha-amylase enzyme.

In some cases, the alpha-amylase enzyme comprises 0.1 percent to 1 percent of the alpha-amylase enzyme to a weight of the High Moisture Extrusion extrudate, the enzymatic treatment comprises a neutral protease enzyme and an alpha-amylase enzyme and a ratio of the neutral protease enzyme to the alpha-amylase enzyme may be from 1 to 0.25 percent and 1 to 1.

In some cases, the automatic and dynamic enzymatic treatment for texturizing and tenderizing the plant protein meat analog food product further comprises increasing the baseline plant protein meat analog food texture to an optimized plant protein meat analog food texture, the optimized plant protein meat analog food texture resulting optimized animal meat-like mouthfeel and taste for the consumers of the plant protein meat analog food product, the automatic and dynamic enzymatic treatment for texturizing and tenderizing the plant protein meat analog food product further comprises decreasing the baseline plant protein meat analog food texture to an optimized plant protein meat analog food texture, the optimized plant protein meat analog food texture resulting optimized animal meat-like mouthfeel and taste for the consumers of the plant protein meat analog food product and the automatic and dynamic enzymatic treatment for texturizing and tenderizing the plant protein meat analog food product further comprises increasing the baseline plant protein meat analog food tenderness to an optimized plant protein meat analog food tenderness, the optimized plant protein meat analog food tenderness resulting in optimized animal meat-like mouthfeel and taste for the consumers of the plant protein meat analog food product.

In some cases, the automatic and dynamic enzymatic treatment for texturizing and tenderizing the plant protein meat analog food product further comprises decreasing the baseline plant protein meat analog food tenderness to an optimized plant protein meat analog food tenderness, the optimized plant protein meat analog food tenderness resulting in optimized animal meat-like mouthfeel and taste for the consumers of the plant protein meat analog food product (e.g., as determined by Sensory Test Report).

In some cases, the one or more computing platforms 202, may be communicatively coupled to the remote platform(s) 204. In some cases, the communicative coupling may include communicative coupling through a networked environment 210. The networked environment 210 may be a radio access network, such as LTE or 5G, a local area network (LAN), a wide area network (WAN) such as the Internet, or wireless LAN (WLAN), for example. It will be appreciated that this is not intended to be limiting, and that the scope of this disclosure includes implementations in which one or more computing platforms 202 and remote platform(s) 204 may be operatively linked via some other communication coupling. The one or more computing platforms 202 may be configured to communicate with the networked environment 210 via wireless or wired connections. In addition, in an embodiment, the one or more computing platforms 202 may be configured to communicate directly with each other via wireless or wired connections. Examples of one or more computing platforms 202 may include, but is not limited to, smartphones, wearable devices, tablets, laptop computers, desktop computers, Internet of Things (IoT) device, or other mobile or stationary devices. In an embodiment, system 100 may also include one or more hosts or servers, such as the one or more remote platforms 204 connected to the networked environment 210 through wireless or wired connections. According to one embodiment, remote platforms 204 may be implemented in or function as base stations (which may also be referred to as Node Bs or evolved Node Bs (eNBs)). In other embodiments, remote platforms 204 may include web servers, mail servers, application servers, etc. According to certain embodiments, remote platforms 204 may be standalone servers, networked servers, or an array of servers.

The one or more computing platforms 202 may include one or more processors 212 for processing information and executing instructions or operations. One or more processors 212 may be any type of general or specific purpose processor. In some cases, multiple processors 212 may be utilized according to other embodiments. In fact, the one or more processors 212 may include one or more of general-purpose computers, special purpose computers, microprocessors, digital signal processors (DSPs), field-programmable gate arrays (FPGAs), application-specific integrated circuits (ASICs), and processors based on a multi-core processor architecture, as examples. In some cases, the one or more processors 212 may be remote from the one or more computing platforms 202, such as disposed within a remote platform like the one or more remote platforms 204 of FIG. 2.

The one or more processors 212 may perform functions associated with the operation of system 100 which may include, for example, precoding of antenna gain/phase parameters, encoding and decoding of individual bits forming a communication message, formatting of information, and overall control of the one or more computing platforms 202, including processes related to management of communication resources.

The one or more computing platforms 202 may further include or be coupled to a memory 214 (internal or external), which may be coupled to one or more processors 212, for storing information and instructions that may be executed by one or more processors 212. Memory 214 may be one or more memories and of any type suitable to the local application environment, and may be implemented using any suitable volatile or nonvolatile data storage technology such as a semiconductor-based memory device, a magnetic memory device and system, an optical memory device and system, fixed memory, and removable memory. For example, memory 214 can consist of any combination of random-access memory (RAM), read only memory (ROM), static storage such as a magnetic or optical disk, hard disk drive (HDD), or any other type of non-transitory machine or computer readable media. The instructions stored in memory 214 may include program instructions or computer program code that, when executed by one or more processors 212, enable the one or more computing platforms 202 to perform tasks as described herein.

In some embodiments, one or more computing platforms 202 may also include or be coupled to one or more antennas for transmitting and receiving signals and/or data to and from one or more computing platforms 202. The one or more antennas may be configured to communicate via, for example, a plurality of radio interfaces that may be coupled to the one or more antennas. The radio interfaces may correspond to a plurality of radio access technologies including one or more of LTE, 5G, WLAN, Bluetooth, near field communication (NFC), radio frequency identifier (RFID), ultrawideband (UWB), and the like. The radio interface may include components, such as filters, converters (for example, digital-to-analog converters and the like), mappers, a Fast Fourier Transform (FFT) module, and the like, to generate symbols for a transmission via one or more downlinks and to receive symbols (for example, via an uplink).

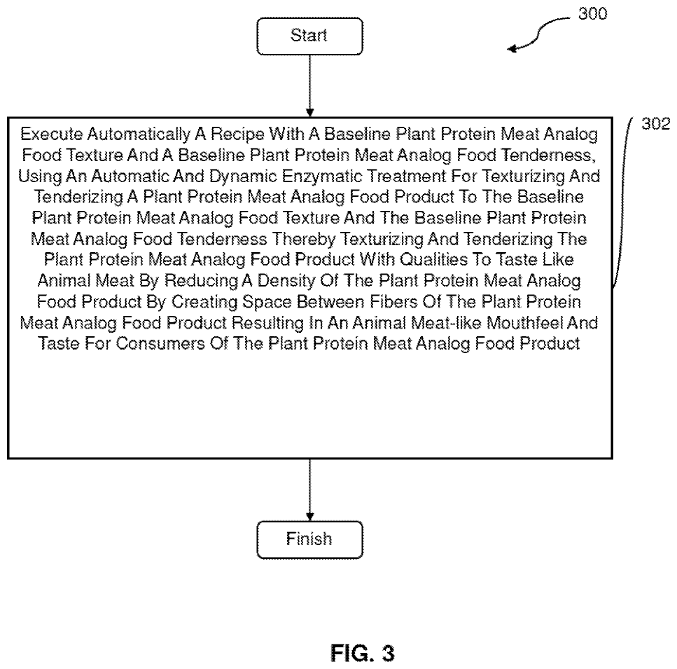

FIG. 3 illustrates an example flow diagram of a method 300, according to one embodiment. The method 300 may include executing automatically, using an electronic sensor system, a recipe with a baseline plant protein meat analog food texture and a baseline plant protein meat analog food tenderness, using an automatic and dynamic enzymatic treatment for texturizing and tenderizing a plant protein meat analog food product to the baseline plant protein meat analog food texture and the baseline plant protein meat analog food tenderness thereby texturizing and tenderizing the plant protein meat analog food product with qualities to taste like animal meat by reducing a density of the plant protein meat analog food product by creating space between fibers of the plant protein meat analog food product resulting in an animal meat-like mouthfeel and taste for consumers of the plant protein meat analog food product at block 302.

In some cases, the method 300 may be performed by one or more hardware processors, such as the processors 214 of FIG. 2, configured by machine-readable instructions, such as the machine-readable instructions 206 of FIG. 2. In this aspect, the method 300 may be configured to be implemented by the modules. The modules may include one or more of recipe executing module 208, and/or other modules as discussed above in FIG. 2.

### Support Examples

Example 1 includes a system comprising: executing automatically, using an electronic sensor system, a recipe with a baseline plant protein meat analog food texture and a baseline plant protein meat analog food tenderness, using an automatic and dynamic enzymatic treatment for texturizing and tenderizing a plant protein meat analog food product to the baseline plant protein meat analog food texture and the baseline plant protein meat analog food tenderness thereby texturizing and tenderizing the plant protein meat analog food product with qualities to taste like animal meat by reducing a density of the plant protein meat analog food product by creating space between fibers of the plant protein meat analog food product resulting in an animal meat-like mouthfeel and taste for consumers of the plant protein meat analog food product. For example, FIG. 1A shows an extruder system that is a system used for texturizing and tenderizing plant protein meat analog food products with animal meat-like qualities using an enzymatic treatment of a High Moisture Extrusion (HME) extrudate by producing the High Moisture Extrusion extrudate 165 (e.g., plant protein meat analog food product that is treated with an enzymatic treatment for texturizing and tenderizing with animal meat-like qualities). Furthermore, FIG. 1B illustrates a top view of a system for vacuum cooking the High Moisture Extrusion (HME) extrudate 165 with the enzymatic treatment.

Example 2 includes the system of example(s) 1 and/or some other example(s) herein, wherein the enzymatic treatment comprises a neutral protease enzyme.

Proteases.

In various embodiments, FoodPro® PNL (i.e., a neutral protease enzyme preparation derived from Bacillus amyloliquefaciens) is used at from 0.25% to 1% (w/w) enzyme to HME weight. In some instances, 1% is a very effective on the High Moisture Extrusion extrudate 165 (e.g., plant protein meat analog food product that is treated with an enzymatic treatment for texturizing and tenderizing with animal meat-like qualities).

Example 2 includes the method of example(s) 1 and/or some other example(s) herein, wherein the enzymatic treatment comprises a neutral protease enzyme and an alpha-amylase enzyme (e.g., FoodPro® PNL and FoodPro® ALT).

Amylases.

In various embodiments, FoodPro® ALT (an alpha-amylase enzyme preparation derived from Bacillus subtilis is used at 0.1% to 0.1% (w/w)—enzyme to HME weight—and is very effective on the High Moisture Extrusion extrudate 165 (e.g., plant protein meat analog food product that is treated with an enzymatic treatment for texturizing and tenderizing with animal meat-like qualities).

As these two components (e.g., FoodPro® PNL and FoodPro® ALT) are working on different parts of the High Moisture Extrusion extrudate 165 (e.g., plant protein meat analog food product that is treated with an enzymatic treatment for texturizing and tenderizing with animal meat-like qualities), the present technology uses a combination of these two enzymes (i.e., FoodPro® PNL and FoodPro® ALT) to target different part of the High Moisture Extrusion extrudate 165 to obtain a more tender product.

In various embodiments, the most effective combination is 1% of FoodPro® PNL protease and 0.7% of FoodPro® ALT Amylase. The final product is very tender and soft which gives a real meat's bite feeling according to our tasters and sensory panel. In some embodiments, the best ratio of FoodPro® PNL to FoodPro® ALT is; 1.43.

Working Conditions.

Working with enzymes, needs some precautions and safety measurement accruing to their SDS data sheet that are incorporated herein by reference for all purposes. Standard safety precautions against are used including working under a hood with a mask when handling the enzymes. In some embodiments, the best activation temperature for enzyme on the High Moisture Extrusion extrudate 165 for both FoodPro® PNL and FoodPro® ALT is 55° C. For example, the temperature was maintained at 55° C. for seven minutes while tumbling the High Moisture Extrusion extrudate 165 with water and enzymes. The temperature was then increased to greater than 90° C. for ten min to deactivate the enzyme during our cooking process. Both FoodPro® PNL and FoodPro® ALT get deactivated at greater than 90° C.

Example 3 includes the system of example(s) 2 and/or some other example(s) herein, wherein the neutral protease enzyme comprises 0.25 percent to 1 percent of the neutral protease enzyme to a weight of the High Moisture Extrusion extrudate.

Example 4 includes the system of example(s) 1 and/or some other example(s) herein, wherein the enzymatic treatment comprises an alpha-amylase enzyme.

Example 5 includes the system of example(s) 4 and/or some other example(s) herein, wherein the alpha-amylase enzyme comprises 0.1 percent to 1 percent of the alpha-amylase enzyme to a weight of the High Moisture Extrusion extrudate.

Example 6 includes the system of example(s) 1 and/or some other example(s) herein, wherein the enzymatic treatment comprises a neutral protease enzyme and an alpha-amylase enzyme.

Example 7 includes the system of example(s) 6 and/or some other example(s) herein, wherein a ratio of the neutral protease enzyme to the alpha-amylase enzyme is from 1 to 0.25 percent and 1 to 1.

Example 8 includes the system of example(s) 1 and/or some other example(s) herein, wherein the automatic and dynamic enzymatic treatment for texturizing and tenderizing the plant protein meat analog food product further comprises increasing the baseline plant protein meat analog food texture to an optimized plant protein meat analog food texture, the optimized plant protein meat analog food texture resulting optimized animal meat-like mouthfeel and taste for the consumers of the plant protein meat analog food product.

Example 9 includes the system of example(s) 1 and/or some other example(s) herein, wherein the automatic and dynamic enzymatic treatment for texturizing and tenderizing the plant protein meat analog food product further comprises decreasing the baseline plant protein meat analog food texture to an optimized plant protein meat analog food texture, the optimized plant protein meat analog food texture resulting optimized animal meat-like mouthfeel and taste for the consumers of the plant protein meat analog food product.

Example 10 includes the system of example(s) 1 and/or some other example(s) herein, wherein the automatic and dynamic enzymatic treatment for texturizing and tenderizing the plant protein meat analog food product further comprises increasing the baseline plant protein meat analog food tenderness to an optimized plant protein meat analog food tenderness, the optimized plant protein meat analog food tenderness resulting in optimized animal meat-like mouthfeel and taste for the consumers of the plant protein meat analog food product.

Example 11 includes the system of example(s) 1 and/or some other example(s) herein, wherein the automatic and dynamic enzymatic treatment for texturizing and tenderizing the plant protein meat analog food product further comprises decreasing the baseline plant protein meat analog food tenderness to an optimized plant protein meat analog food tenderness, the optimized plant protein meat analog food tenderness resulting in optimized animal meat-like mouthfeel and taste for the consumers of the plant protein meat analog food product.

Exemplary Sensory Test Report (Subject: Enzyme Patent Test Results).

An exemplary Sensory Test Report was executed on Nov. 18, 2021.

Background.

In various embodiments the present technology uses enzymes to increase the tenderness of the High Moisture Extrusion extrudate 165 (e.g., plant protein meat analog food product that is treated with an enzymatic treatment for texturizing and tenderizing with animal meat-like qualities; i.e., chicken-like products such as LikeMeat Chick'n Pieces. In various embodiments, the present technology uses Proteases PNL at 1% and Amylases ALT at 0.7% to noticeably increase tenderness and texture of the High Moisture Extrusion extrudate 165 (e.g., plant protein meat analog food product that is treated with an enzymatic treatment for texturizing and tenderizing with animal meat-like qualities. To validate this result, the Sensory Test Report was conducted. A directional, paired comparison test was selected to ensure participants were focused exclusively on the tenderness of the meat.

Testing Method.

A double-blind, directional paired-comparison test was conducted with nine participants in replicate to determine if there was a noticeable difference in tenderness between LikeMeat Chick'n Pieces with and without enzyme.

In an exemplary test, nine participants were enrolled with a range of experience in performing sensory tests.

Product Information:

| | Format | Enzyme | Blinding codes |
|---|---|---|---|
| Control | LikeMeat | No | 601, 521 |
| | Chick'n Pieces | | |
| Test | LikeMeat | Yes | 832, 632 |
| | Chick'n Pieces | | |

Preparation & Serving:

Benchtop samples were being prepared a day before tasting and kept frozen overnight (mimicking the condition in production line). At the time of the tasting, samples prepared as written on the packaging (add a little oil on a frying pan on a medium heat for 6 min until the internal temp reach to 165° F. Both samples' series (control and test) prepared at the sample time to reduce the variabilities and keep the condition the same for both samples. Samples were served immediately after preparation to tasters and each tasters got four pieces of each sample to evaluate the texture according to the questioner.

Samples served on a white plate labeled with 3-digit code.

### RESULTS & CONCLUSION

| | Number selected | Number selected |
|---|---|---|
| | as “more | as “more |
| | tender” Replicate 1 | tender” Replicate 2 |
| Control (without enzyme) | 0 | 0 |
| Test (with enzyme) | 9 | 9 |

All participants were able to correctly identify the test sample as being more tender in both replicates. The results from this test indicate that the enzyme is increasing the perception of tenderness of LikeMeat Chick'n Pieces made on the benchtop.

While this technology is susceptible of embodiments in many different forms, there is shown in the drawings and has been described in detail several specific embodiments with the understanding that the present disclosure is to be considered as an exemplification of the principles of the technology and is not intended to limit the technology to the embodiments illustrated.

Although the terms first, second, etc. may be used herein to describe various elements, components, regions, layers and/or sections, these elements, components, regions, layers and/or sections should not necessarily be limited by such terms. These terms are only used to distinguish one element, component, region, layer or section from another element, component, region, layer, or section. Thus, a first element, component, region, layer, or section discussed below could be termed a second element, component, region, layer, or section without departing from the teachings of the present disclosure.

The terminology used herein is for the purpose of describing particular embodiments only and is not intended to be necessarily limiting of the disclosure. As used herein, the singular forms “a,” “an” and “the” are intended to include the plural forms as well, unless the context clearly indicates otherwise. The terms “comprises,” “includes” and/or “comprising,” “including” when used in this specification, specify the presence of stated features, integers, steps, operations, elements, and/or components, but do not preclude the presence or addition of one or more other features, integers, steps, operations, elements, components, and/or groups thereof.

Example embodiments of the present disclosure are described herein with reference to illustrations of idealized embodiments (and intermediate structures) of the present disclosure. As such, variations from the shapes of the illustrations as a result, for example, of manufacturing techniques and/or tolerances, are to be expected. Thus, the example embodiments of the present disclosure should not be construed as necessarily limited to the particular shapes of regions illustrated herein, but are to include deviations in shapes that result, for example, from manufacturing.

Unless otherwise defined, all terms (including technical and scientific terms) used herein have the same meaning as commonly understood by one of ordinary skill in the art to which this disclosure belongs. The terms, such as those defined in commonly used dictionaries, should be interpreted as having a meaning that is consistent with their meaning in the context of the relevant art and should not be interpreted in an idealized and/or overly formal sense unless expressly so defined herein.

Furthermore, relative terms such as “below,” “lower,” “above,” and “upper” may be used herein to describe one element's relationship to another element as illustrated in the accompanying drawings. Such relative terms are intended to encompass different orientations of illustrated technologies in addition to the orientation depicted in the accompanying drawings. For example, if a device in the accompanying drawings is turned over, then the elements described as being on the “lower” side of other elements would then be oriented on “upper” sides of the other elements. Similarly, if the device in one of the figures is turned over, elements described as “below” or “beneath” other elements would then be oriented “above” the other elements. Therefore, the example terms “below” and “lower” can, therefore, encompass both an orientation of above and below.

The description of the present disclosure has been presented for purposes of illustration and description but is not intended to be exhaustive or limited to the present disclosure in the form disclosed. Many modifications and variations will be apparent to those of ordinary skill in the art without departing from the scope and spirit of the present disclosure. Exemplary embodiments were chosen and described in order to best explain the principles of the present disclosure and its practical application, and to enable others of ordinary skill in the art to understand the present disclosure for various embodiments with various modifications as are suited to the particular use contemplated.

While various embodiments have been described above, it should be understood that they have been presented by way of example only, and not limitation. The descriptions are not intended to limit the scope of the technology to the particular forms set forth herein. Thus, the breadth and scope of a preferred embodiment should not be limited by any of the above-described exemplary embodiments. It should be understood that the above description is illustrative and not restrictive. To the contrary, the present descriptions are intended to cover such alternatives, modifications, and equivalents as may be included within the spirit and scope of the technology as defined by the appended claims and otherwise appreciated by one of ordinary skill in the art. The scope of the technology should, therefore, be determined not with reference to the above description, but instead should be determined with reference to the appended claims along with their full scope of equivalents.

What is claimed is:

1. A system for texturizing and tenderizing plant protein meat analog food products with animal meat-like qualities using an enzymatic treatment of a High Moisture Extrusion (HME) extrudate, the system comprising:
a high moisture extrusion (HME) system including using an automatic and dynamic enzymatic treatment according to a recipe for texturizing and tenderizing a plant protein meat analog food product, the high moisture extrusion (HME) system comprising:
a dry feed system, the dry feed system feeding plant protein into the high moisture extrusion (HME) system;
a water feed system, the water feed system feeding water into the high moisture extrusion (HME) system to mix with the plant protein;
a barrel system for mixing the plant protein and the water, the barrel system comprising:
a shaft;
at least one screw controlled by the shaft; and
a heating system;
a cooling die, the cooling die being maintained at a baseline cooling die temperature by the heating system and producing a High Moisture Extrusion (HME) extrudate;
a vacuum tumbler for cooking the High Moisture Extrusion (HME) extrudate to the plant protein meat analog food product;
an enzyme pump, the enzyme pump automatically and dynamically adding the enzymatic treatment according to the recipe to the vacuum tumbler with the High Moisture Extrusion (HME) extrudate;
an electronic sensor system, the electronic sensor system electronically connected to the dry feed system, the water feed system, the barrel system, the cooling die, the vacuum tumbler, the enzyme pump; and
a main operation panel, the main operation panel being electronically connected to the electronic sensor system, the main operation panel comprising:
at least one processor; and
a memory storing processor-executable instructions, wherein the at least one processor is configured to implement the following operations for the automatic and dynamic enzymatic treatment for texturizing and tenderizing the plant protein meat analog food product upon executing the processor-executable instructions:
executing automatically, using the electronic sensor system, the recipe with a baseline plant protein meat analog food texture and a baseline plant protein meat analog food tenderness, using the automatic and dynamic enzymatic treatment for texturizing and tenderizing the plant protein meat analog food product to the baseline plant protein meat analog food texture and the baseline plant protein meat analog food tenderness thereby texturizing and tenderizing the plant protein meat analog food product with qualities to taste like animal meat by reducing a density of the plant protein meat analog food product by creating space between fibers of the plant protein meat analog food product resulting in an animal meat-like mouthfeel and taste for consumers of the plant protein meat analog food product.

2. The system of claim 1, wherein the enzymatic treatment comprises a neutral protease enzyme.

3. The system of claim 2, wherein the neutral protease enzyme comprises 0.25 percent to 1 percent (w/w) of the neutral protease enzyme to a weight of the High Moisture Extrusion (HME) extrudate.

4. The system of claim 1, wherein the enzymatic treatment comprises an alpha-amylase enzyme.

5. The system of claim 4, wherein the alpha-amylase enzyme comprises 0.1 percent to 1 percent (w/w) of the alpha-amylase enzyme to a weight of the High Moisture Extrusion (HME) extrudate.

6. The system of claim 1, wherein the enzymatic treatment comprises a neutral protease enzyme and an alpha-amylase enzyme.

7. The system of claim 6, wherein a ratio of the neutral protease enzyme to the alpha-amylase enzyme is from 1 to 0.25 percent and 1 to 1.

8. The system of claim 1, wherein the automatic and dynamic enzymatic treatment for texturizing and tenderizing the plant protein meat analog food product further comprises increasing the baseline plant protein meat analog food texture to an optimized plant protein meat analog food texture, the optimized plant protein meat analog food texture resulting optimized animal meat-like mouthfeel and taste for the consumers of the plant protein meat analog food product.

9. The system of claim 1, wherein the automatic and dynamic enzymatic treatment for texturizing and tenderizing the plant protein meat analog food product further comprises decreasing the baseline plant protein meat analog food texture to an optimized plant protein meat analog food texture, the optimized plant protein meat analog food texture resulting optimized animal meat-like mouthfeel and taste for the consumers of the plant protein meat analog food product.

10. The system of claim 1, wherein the automatic and dynamic enzymatic treatment for texturizing and tenderizing the plant protein meat analog food product further comprises increasing the baseline plant protein meat analog food tenderness to an optimized plant protein meat analog food tenderness, the optimized plant protein meat analog food tenderness resulting in optimized animal meat-like mouthfeel and taste for the consumers of the plant protein meat analog food product.

11. The system of claim 1, wherein the automatic and dynamic enzymatic treatment for texturizing and tenderizing the plant protein meat analog food product further comprises decreasing the baseline plant protein meat analog food tenderness to an optimized plant protein meat analog food tenderness, the optimized plant protein meat analog food tenderness resulting in optimized animal meat-like mouthfeel and taste for the consumers of the plant protein meat analog food product.

12. A method for texturizing and tenderizing plant protein meat analog food products with animal meat-like qualities using an enzymatic treatment of a High Moisture Extrusion (HME) extrudate, the method comprising:
executing automatically, using an electronic sensor system, a recipe with a baseline plant protein meat analog food texture and a baseline plant protein meat analog food tenderness, using an automatic and dynamic enzymatic treatment for texturizing and tenderizing a plant protein meat analog food product to the baseline plant protein meat analog food texture and the baseline plant protein meat analog food tenderness thereby texturizing and tenderizing the plant protein meat analog food product with qualities to taste like animal meat by reducing a density of the plant protein meat analog food product by creating space between fibers of the plant protein meat analog food product resulting in an animal meat-like mouthfeel and taste for consumers of the plant protein meat analog food product.

13. The method of claim 12, wherein the enzymatic treatment comprises a neutral protease enzyme.

14. The method of claim 13, wherein the neutral protease enzyme comprises 0.25 percent to 1 percent (w/w) of the neutral protease enzyme to a weight of the High Moisture Extrusion (HME) extrudate.

15. The method of claim 12, wherein the enzymatic treatment comprises an alpha-amylase enzyme.

16. The method of claim 15, wherein the alpha-amylase enzyme comprises 0.1 percent to 1 percent (w/w) of the alpha-amylase enzyme to a weight of the High Moisture Extrusion (HME) extrudate.

17. The method of claim 12, wherein the enzymatic treatment comprises a neutral protease enzyme and an alpha-amylase enzyme.

18. The method of claim 17, wherein a ratio of the neutral protease enzyme to the alpha-amylase enzyme is from 1 to 0.25 percent and 1 to 1.

19. A non-transient computer-readable storage medium comprising instructions being executable by one or more processors to perform operations for texturizing and tenderizing plant protein meat analog food products with animal meat-like qualities using an enzymatic treatment of a High Moisture Extrusion (HME) extrudate, the operations comprising:
executing automatically, using an electronic sensor system, a recipe with a baseline plant protein meat analog food texture and a baseline plant protein meat analog food tenderness, using an automatic and dynamic enzymatic treatment for texturizing and tenderizing a plant protein meat analog food product to the baseline plant protein meat analog food texture and the baseline plant protein meat analog food tenderness thereby texturizing and tenderizing the plant protein meat analog food product with qualities to taste like animal meat by reducing a density of the plant protein meat analog food product by creating space between fibers of the plant protein meat analog food product resulting in an animal meat-like mouthfeel and taste for consumers of the plant protein meat analog food product.

20. The non-transient computer-readable storage medium of claim 19, wherein the enzymatic treatment comprises a neutral protease enzyme and an alpha-amylase enzyme.
